(19) United States
(12) Patent Application Publication
(10) Pub. No.: US 2026/0294781 A1
(43) Pub. Date: Oct. 1, 2026

(54) A COSMETIC COMPOSITION

(71) Applicants: Conopco, Inc., d/b/a UNILEVER, Hoboken, NJ (US); UNILEVER IP HOLDINGS B.V., Wageningen (NL)

(72) Inventors: Shufan HAN, Shanghai (CN); Xiaoke LI, Shanghai (CN); Renjiang LIU, Shanghai (CN)

(73) Assignee: Conopco, Inc., d/b/a UNILEVER, Hoboken, NJ (US)

(21) Appl. No.: 19/479,055

(22) PCT Filed: Apr. 10, 2024
(86) PCT No.: PCT/EP2024/059685

§ 371 Date: Oct. 27, 2025

(30) Foreign Application Priority Data
Apr. 27, 2023 (WO) PCT/CN2023/091166
Jun. 5, 2023 (EP) 23177165.0

(51) Int. Cl. A61K 8/81; A61K 8/04; A61K 8/20; A61K 8/27; A61Q 15/00; A61Q 17/00

(52) U.S. Cl. CPC A61K 8/8147; A61K 8/046; A61K 8/20; A61K 8/27; A61Q 15/00; A61Q 17/005; A61K 2800/30

(57) ABSTRACT

The present invention is in the field of cosmetic compositions, in particular, compositions which can provide sweat management benefit. Disclosed is an aqueous cosmetic composition comprising: (i) a polymer selected from polyacrylic acid, polyacrylate, poly methacrylic acid and polymethacrylate; (ii) a water-soluble metal salt wherein the metal is selected from calcium, magnesium, and zinc; wherein the molecular weight of the polymer is lower than 9500 g/mol; wherein pH of the composition is from 3 to 5.5; wherein the composition comprises at least 1% of said polymer by weight of the composition.

### FIELD OF THE INVENTION

The present invention is in the field of cosmetic compositions, particularly compositions which can provide sweat management benefit, which is capable of preventing sweat from rapidly being released, absorbing or managing excess sweat.

### BACKGROUND OF THE INVENTION

The present invention relates to cosmetic compositions, particularly compositions which can provide sweat management benefit. The compositions are used to control or manage moisture (or excess sweat) on the skin surface upon topical application of the compositions to the body, particularly to the underarm regions of the human body viz, the axilla, and sometimes even on the upper part of the body near the chest. Such composition can help prevent sweat from rapidly being released, absorb or manage excess sweat on skin surface upon topical application of the compositions to the body.

### SUMMARY OF THE INVENTION

The present inventors have surprisingly observed that an aqueous cosmetic composition comprising certain amount of specific polymer selected from polyacrylic acid, polyacrylate, poly methacrylic acid and polymethacrylate which has a low molecular weight, and certain amount of a water-soluble metal salt wherein the metal is selected from calcium, magnesium, and zinc at certain level of pH can lead to sweat management benefit. The specific polymer can be stable in an aqueous composition with specific water-soluble metal salt at certain level of pH. In addition to being stable, the combination of specific polymer and specific water-soluble metal salt is capable of forming precipitation/aggregation when they contact with aqueous saline media, which is equivalent to sweat, thereby is capable of preventing sweat from rapidly being released, absorbing or managing excess sweat. In this manner, such composition can control or manage moisture (or excess sweat) on the desired skin surface.

In accordance with a first aspect is disclosed an aqueous cosmetic composition comprising:
(i) a polymer selected from polyacrylic acid, polyacrylate, poly methacrylic acid and polymethacrylate;
(ii) a water-soluble metal salt wherein the metal is selected from calcium, magnesium, and zinc;
wherein the molecular weight of the polymer is lower than 9500 g/mol; wherein pH of the composition is from 3 to 5.5; wherein the composition comprises at least 1% of said polymer by weight of the composition.

In accordance with a second aspect is disclosed a method of preventing sweat from rapidly being released, absorbing or managing excess sweat comprising a step of topical application of the composition of the first aspect on to the desired skin surface.

In accordance with a third aspect is disclosed use of the composition of the first aspect for preventing sweat from rapidly being released, absorbing or managing excess sweat on desired skin surface.

All other aspects of the present invention will more readily become apparent upon considering the detailed description and examples which follow.

Except in the examples, or where otherwise explicitly indicated, all numbers in this description indicating amounts of material or conditions of reaction, physical properties of materials and/or use may optionally be understood as modified by the word “about”.

All amounts are by weight of the composition, unless otherwise specified.

It should be noted that in specifying any ranges of values, any particular upper value can be associated with any particular lower value.

For the avoidance of doubt, the word “comprising” is intended to mean “including” but not necessarily “consisting of” or “composed of”. In other words, the listed steps or options need not be exhaustive.

The disclosure of the invention as found herein is to be considered to cover all embodiments as found in the claims as being multiply dependent upon each other irrespective of the fact that claims may be found without multiple dependency or redundancy.

Where a feature is disclosed with respect to a particular aspect of the invention (for example a composition of the invention), such disclosure is also to be considered to apply to any other aspect of the invention (for example a method of the invention) mutatis mutandis.

### DETAILED DESCRIPTION OF THE INVENTION

By “A cosmetic composition” as used herein, is meant to include a composition for topical application to the skin of mammals, especially humans. Such a composition is preferably of the leave-on type. By a leave-on composition is meant a composition that is applied to the desired skin surface and left on for one minute to 24 hours after which it may be wiped or rinsed off with water, usually during the regular course of personal washing. The composition may also be formulated into a product which is applied to a human body for improving the appearance, cleansing, odor control or general aesthetics. The composition of the present invention can be in the form of a liquid, lotion, cream, gel or stick form and may be delivered through a roll-on device or using an aerosol can which contains a propellant. “Skin” as used herein is meant to include skin on any part of the body (e.g., neck, chest, back, arms, underarms, hands, legs, buttocks and scalp) especially the underarms. The cosmetic composition in accordance with the present invention is sweat management composition which is used for providing sweat management benefit which is preventing sweat from rapidly being released, absorbing or managing excess sweat on the skin surface.

By “water-soluble” as used herein, is meant to be a salt that has a solubility in water at 20° C. and 1 atm of greater than 1 g salt/100 ml water.

“Molecular weight”, as used herein, refers to the weight average molecular weight of a given polymer. The weight average molecular weight (WAVG MW) of a given polymer can be determined by SEC (Size Exclusion Chromatography) analysis using absolute calibration (universal calibration).

### Polymer

The polymer in accordance with this invention is selected from polyacrylic acid, polyacrylate, poly methacrylic acid and polymethacrylate. It is preferred that the polymer is polymethacrylate. Such polymers are water-soluble and not cross-linked. A polyacrylate is a salt or ester of polyacrylic acid. When the polymer is a polyacrylate, it is preferably selected from sodium polyacrylate, potassium polyacrylate, ammonium polyacrylate, and lithium polyacrylate, more preferably it is sodium polyacrylate.

A polymethacrylate is a salt or ester of poly methacrylic acid. When the polymer is a polymethacrylate, it is preferably selected from sodium polymethacrylate, potassium polymethacrylate, ammonium polymethacrylate, and lithium polymethacrylate, more preferably it is sodium polymethacrylate.

It is more preferred that the polymer is selected low molecular weight water soluble acrylic/methacrylic acid polymer, and sodium salt of low molecular weight water soluble acrylic/methacrylic acid polymer.

The polymer in accordance with this invention has a molecular weight lower than 9500 g/mol, preferably lower than 9000 g/mol, more preferably not higher than 8000 g/mol. It is preferred that the polymer has a molecular weight from 2000 to 9000 g/mol, more preferably from 2000 to 8000 g/mol, furthermore preferably from 2000 to 6000 g/mol.

The composition in accordance with this invention comprises at least 1% of the polymer, preferably at least 2%, more preferably at least 4% of the polymer by weight of the composition.

It is preferred that the composition comprises from 1 to 20% of the polymer, more preferably from 2 to 15%, furthermore preferably from 4 to 10% by weight of the

### Water-Soluble Metal Salt

The water-soluble metal salt in accordance with this invention is bivalent metal salt wherein the metal is selected from calcium, magnesium and zinc.

Water-soluble calcium salt can be one of calcium chloride, calcium gluconate, calcium nitrate, calcium bromide, calcium formate, calcium lactate, calcium propanoate, calcium acetate, and calcium dobesilate.

Water-soluble magnesium salt can be one of magnesium chloride, magnesium nitrate, magnesium sulphate, magnesium citrate, magnesium gluconate, magnesium acetate, magnesium lactate, and magnesium malate.

Water-soluble zinc salt can be one of zinc chloride, zinc nitrate, zinc citrate, zinc sulphate, and zinc gluconate.

It is preferred that the composition comprises at least 0.5%, more preferably at least 1%, furthermore preferably at least 1.5% of the water-soluble metal salt by weight of the composition.

It is preferred that the composition comprises from 0.5 to 10%, more preferably from 1 to 8%, furthermore preferably from 1.5 to 5% of the water-soluble metal salt by weight of the composition.

### pH of Compositions

The pH of the composition of the present invention is from 3 to 5.5, preferably from 3 to 5, more preferably 3.5 to 4.5.

### Other Ingredients

Other components commonly included in cosmetic compositions may also be incorporated in the compositions of the present invention. Such components include skin care agents such as emollients, humectants and skin barrier promoters; skin appearance modifiers such as skin lightening agents and skin smoothing agents; anti-microbial agents, in particular organic anti-microbial agents, and preservatives.

The composition may include skin actives selected from a group consisting of alpha-hydroxy acids, mandelic acid, methylxanthines, alkaloids and caffeine. It is preferred free of parabens.

The compositions of the invention are applied cosmetically and topically to the skin, broadly speaking, by one of two methods. Different consumers prefer one method or the other. In one method, sometimes called a contact method, the composition is wiped across the surface of the skin, depositing a fraction of the composition as it passes. In the second method, sometimes called the non-contact method, the composition is sprayed from a dispenser held proximate to the skin, often in an area of about 10 to 20 cm². The spray can be developed by mechanical means of generating pressure on the contents of a dispenser, such as a pump or a squeezable sidewall or by internally generated pressure arising from a fraction of a liquefied propellant volatilizing, the dispenser commonly being called an aerosol.

There are broadly speaking two classes of contact compositions, one of which is liquid and usually applied using a roll-on dispenser or possibly absorbed into or onto a wipe, and in the second of which the active is distributed within a carrier liquid that forms a continuous phase that has been gelled. In one variation, the carrier fluid comprises a solvent for the active and in a second variation, the active remains a particulate solid that is suspended in an oil, usually a blend of oils.

The composition of the invention comprises an aqueous cosmetically acceptable carrier. The term aqueous means that the composition of the invention comprises water as the main carrier or that water forms a major part of the carrier. In such cases, other solvents and ingredients other than water may also be present. It is preferred that the composition comprises from 5 to 98% of water, preferably from 15 to 95%, more preferably from 50 to 90%, furthermore preferably from 70 to 90% by weight of the composition.

It is preferred that the composition of the invention is in the form of a cream, a spray, a firm solid, a soft solid or is an emulsion packaged in a roll-on applicator.

Alternatively, it is preferred that the composition is in the form of wipes. Wipes here are intended for applying the composition. Wipes are usually made of materials such as polyester, polypropylene, cotton, wood pulp, or rayon fibers formed into sheets. They may be packaged individually, or in small or bulk packaging. They are usually moistened with water, or ethanol or other ingredients that help them work. They may contain other ingredients, such as preservatives to prevent the growth of bacteria and molds.

It is preferred that, when said composition is a spray it comprises a propellant and the composition is in the form of an aerosol.

Further preferably, the composition of the invention is in the form of a roll-on or an aerosol product.

The compositions in accordance with this invention may comprise an antiperspirant active.

However, the composition of the present invention is preferably free from metal based antiperspirant active.

The composition of the present invention is preferably free from antiperspirant active which comprises aluminium or zirconium. And the composition of the present invention is preferably free from antiperspirant active which is zinc based.

Non-aluminum and non-zirconium, non-zinc antiperspirant actives may be present in the composition. Non-aluminum or zirconium, or non-zinc antiperspirant active is preferably selected from glycerol monolaurate plus isostearyl alcohol, chitosan or a salt thereof with a weight average molecular weight of from 250 to 650 kDa, titanium compound chelated by alkanolamine with an acid and a polyhydric alcohol, or cholic acid derivative selected from a hydroxycholic acid or a salt thereof with a multivalent metal salt.

### Stick Compositions

Many different materials have been proposed as gellants for a continuous oil phase, including waxes, small molecule gelling agents and polymers. They each have their advantages and of them, one of the most popular class of gellants is waxes, partly at least due to their ready availability and ease of processing, including in particular linear fatty alcohol wax gellants. A gelled composition is applied topically to skin by wiping it across and in contact with the skin, thereby depositing on the skin a thin film.

The nature of the film depends to a significant extent on the gellant that is employed. Although wax fatty alcohols have been employed as gellants for many years, and are effective for the purpose of gelling, the resultant product is rather ineffective at improving the visual appearance of skin, and in particular underarm skin, to which the composition has been applied. This problem has been solved by including ameliorating materials for example, di or polyhydric humectants and/or a triglyceride oil.

Stick compositions are usually available in the form of a firm solid or a soft solid. Firm solids, as the name indicates, are harder and can be directly applied by way of an applicator, for example, to the underarms. Soft solids also need an applicator which is similar to the firm solids, the difference being that the soft solids are softer and the applicator needs to be designed in order to permit extrusion of the solids through a cap member comprising plurality of orifices and the extruded composition can then be applied to the underarms.

### Roll-on

Liquid compositions that are applicable from a roll-on broadly speaking can be divided into two classes, namely those in which an active is suspended in a hydrophobic carrier, such as a volatile silicone and those in which the active is dissolved in a carrier liquid. The latter has proven to be more popular. There are mainly two sorts of dissolving carrier liquid, namely carriers that are predominantly alcoholic, which is to say the greater part of the dissolving carrier fluid comprises ethanol and the second class in which the carrier liquid is mainly water. The former was very popular because ethanol is a mild bactericide in its own right, but its popularity waned because it stings, especially if the surface onto which the composition has been applied has been damaged or cut, such as can easily arise during shaving or other depilatory actions.

The second class of formulations that is an alternative to alcoholic formulations comprise a dispersion of water-insoluble or very poorly water-soluble ingredients in an aqueous solution of the active. Herein, such compositions will be called emulsions. Roll-on emulsions commonly comprise one or more emulsifiers to maintain a distribution of the water-soluble ingredients.

### Aerosol Compositions

Further alternatively, the composition of the invention is delivered through an aerosol composition which comprises a propellant in addition to the applicable other ingredients described hereinabove. Commonly, the propellant is employed in a weight ratio to the base formulation of from 95:5 to 5:95. Depending on the propellant, in such aerosol compositions the ratio of propellant to base formulation is normally at least 20:80, generally at least 30:70, particularly at least 40:60, and in many formulations, the weight ratio is from 90:10 to 50:50. A ratio range of from 70:30 to 90:10 is sometimes preferred.

Propellants herein generally are one of three classes; (i) low boiling-point gasses liquified by compression, (ii) volatile ethers and (iii) compressed non-oxidising gases.

Class (i) is conveniently a low boiling-point material, typically boiling below −5° C., and often below −15° C., and in particular, alkanes and/or halogenated hydrocarbons. This class of propellant is usually liquefied at the pressure in the aerosol canister and evaporates to generate the pressure to expel the composition out of the canister. Examples of suitable alkanes include particularly propane, butane or isobutane. The class (ii) of propellant comprises a very volatile ether of which the most widely employed ether hitherto is dimethyl ether. This propellant can advantageously be employed at relatively low weight ratio of propellant to base formulation, for example to as low as 5:95. It can also be employed in admixture with, for example, compressible/liquefiable alkane gasses. The class (iii) of propellant comprises compressed non-oxidising gasses, and in particular carbon dioxide or nitrogen. Inert gases like neon are a theoretical alternative.

The composition of the present invention can comprise a wide range of other optional components. The CTFA Personal Care Ingredient Handbook, Second Edition, 1992, which is incorporated by reference herein in its entirety, describes a wide variety of non-limiting personal care and pharmaceutical ingredients commonly used in the skin care industry, which are suitable for use in the compositions of the present invention. Examples include: antioxidants, binders, biological additives, buffering agents, colorants, thickeners, polymers, astringents, fragrance, conditioners, exfoliating agents, pH adjusters, other than the ones already discussed earlier, preservatives, natural extracts, essential oils, skin sensates, skin soothing agents, and skin healing agents.

A preservative is a preferred additional component in compositions of the invention. A preservative serves to reduce or eliminate microbial contamination of compositions of the invention. Preservatives are typically employed at a total level of from 0.05 to 3%, preferably at from 0.1 to 2% and most preferably at from 0.4 to 1%.

Suitable preservatives for use with the present invention include 2-phenoxyethanol, polylysine, iodopropynyl butylcarbamate, C₁-C₃ alkyl parabens, sodium benzoate, caprylyl glycol and EDTA. Particularly preferred preservatives are 2-phenoxyethanol, iodopropynyl butylcarbamate, sodium benzoate, caprylyl glycol and EDTA and especially preferred are 2-phenoxyethanol and iodopropynyl butylcarbamate.

A preferred additional component of compositions of the invention is a fragrance. Suitable materials include conventional perfumes, such as perfume oils and also include so-called deo-perfumes, as described in EP 545,556 and other publications. Levels of incorporation are preferably up to 4% by weight, particularly from 0.1% to 2% by weight, and especially from 0.7% to 1.7% by weight.

An antimicrobial deodorant active is a preferred an additional component in compositions of the invention. Such components serve to reduce or eliminate body odour by reducing or otherwise impeding the function of microbes on the skin of the body responsible for malodour generation.

The antimicrobial deodorant active may also be a preservative for the composition.

When employed, the anti-microbial deodorant agent is typically incorporated into the composition at from 0.01% to 3% and particularly at from 0.03% to 0.5%.

Preferred anti-microbial deodorant agents have a minimum inhibitory concentration (MIC) of 1 mg·ml⁻¹ or less, particularly 200 μg·ml⁻¹ or less, and especially 100 μg·ml⁻¹ or less. The MIC of an anti-microbial agent is the minimum concentration of the agent required to significantly inhibit microbial growth. Inhibition is considered “significant” if an 80% or greater reduction in the growth of an inoculum of Staphylococcus epidermidis is observed, relative to a control medium without an anti-microbial agent, over a period of 16 to 24 hours at 37° C. Details of suitable methods for determining MICs can be found in “Antimicrobial Agents and Susceptibility Testing”, C. Thornsberry, (in “Manual of Clinical Microbiology”, 5^{th} Edition, Ed. A. Balows et al, American Society for Microbiology, Washington D.C., 1991). A particularly suitable method is the Macrobroth Dilution Method as described in Chapter 110 of above publication (pp. 1101-1111) by D. F. Sahm and J. A. Washington II. MICs of anti-microbials suitable for inclusion in the compositions of the invention are triclosan: 0.01-10 μg·ml⁻¹ (J. Regos et al., Dermatologica (1979), 158: 72-79) and farnesol: ca. 25 μg·ml⁻¹ (K. Sawano, T. Sato, and R. Hattori, Proceedings of the 17^{th} IFSCC International Conference, Yokahama (1992) p. 210-232). By contrast ethanol and similar alkanols have MICs of greater than 1 mg·ml⁻¹.

Suitable organic anti-microbials are bactericides, for example quaternary ammonium compounds, like cetyltrimethylammonium salts; chlorhexidine and salts thereof; and diglycerol monocaprate, diglycerol monolaurate, glycerol monolaurate, and similar materials, as described in “Deodorant Ingredients”, S. A. Makin and M. R. Lowry, in “Antiperspirants and Deodorants”, Ed. K. Laden (1999, Marcel Dekker, New York). More preferred anti-microbials for use in the compositions of the invention are polyhexamethylene biguanide salts (also known as polyaminopropyl biguanide salts), an example being Cosmocil CQ™ available from Zeneca PLC, preferably used at up to 1% and more preferably at 0.03% to 0.3% by weight; 2′,4,4′-trichloro,2-hydroxy-diphenyl ether (triclosan), preferably used at up to 1% by weight of the composition and more preferably at 0.05-0.3%; and 3,7,11-trimethyldodeca-2,6,10-trienol (farnesol), preferably used at up to 1% by weight of the composition and more preferably at up to 0.5%.

Other suitable organic antimicrobial agents are transition metal chelators, as described in WO01/52805, for example. Transitional metal chelators having a binding coefficient for iron (III) of greater than 1026, for example diethylenetriaminepentaacetic acid and salts thereof are preferred.

### Method and Use

The present invention also provides for a method of preventing sweat from rapidly being released, absorbing or managing excess sweat comprising a step of topical application of the composition of the first aspect. Preferably, the present invention provides for a method wherein the composition of the first aspect is applied on the underarms. Alternatively, it is preferred that the present invention provides for a method wherein the composition of the first aspect is applied on the skin of body. The present invention also provides for a method wherein topical application of the composition of the first aspect preventing sweat from rapidly being released, absorbing or managing excess sweat on the desired skin surface of the human body. The method in accordance with the invention is preferably non-therapeutic. By non-therapeutic is meant that the method is cosmetic in nature.

The invention also provides for use of the composition of the first aspect for preventing sweat from rapidly being released, absorbing or managing excess sweat on the desired skin surface. Preferably, the desired skin surface is the skin of underarms. Alternatively, it is preferred that the desired skin surface is the skin of body. The use in accordance with the invention is preferably non-therapeutic in nature, more preferably cosmetic in nature.

“Preventing sweat from rapidly being released” as used herein means reducing the rate of release of sweat.

The invention will now be demonstrated with the help of the following non-limiting examples.

### EXAMPLES

### Example 1

This example demonstrated the composition as per the invention are capable of forming an aggregation/precipitation under the test conditions. The compositions according to the formulations detailed in Table 1 are prepared. All ingredients are expressed by weight percent of the total formulation, and as level of active ingredient. The MW of the polymers in the table means weight average molecular weight.

TABLE 1

| | | | | pH (with |
|---|---|---|---|---|
| Samples | Polymer | Metal salt | Water | HCl/NaOH) |
| A | 0.5% sodium | 0.5% calcium | To 100 | 5.00 |
| | polyacrylate | chloride | | |
| | (MW = 5000 g/mol) | | | |
| B | 0.5% sodium | 0.5% calcium | To 100 | 3.69 |
| | polymethacrylate | chloride | | |
| | (MW = 4000-6000 | | | |
| | g/mol) | | | |
| C | 1% sodium | 0.5% calcium | To 100 | 4.61 |
| | polymethacrylate | chloride | | |
| | (MW = 9500) | | | |
| D | 1% sodium | 0.5% calcium | To 100 | 6.16 |
| | polyacrylate | chloride | | |
| | (MW = 5000 g/mol) | | | |
| 1 | 1% sodium | 0.5% calcium | To 100 | 4.57 |
| | polyacrylate | chloride | | |
| | (MW = 5000 g/mol) | | | |
| 2 | 1% sodium | 0.5% calcium | To 100 | 4.93 |
| | polymethacrylate | chloride | | |
| | (MW = 4000-6000 | | | |
| | g/mol) | | | |
| 3 | 4% sodium | 1.68% calcium | To 100 | 4.02 |
| | polyacrylate | chloride | | |
| | (MW = 2100 g/mol) | | | |
| 4 | 4% sodium | 1.68% zinc | To 100 | 3.97 |
| | polyacrylate | sulfate | | |
| | (MW = 5000 g/mol) | | | |
| 5 | 4% sodium | 2.0% calcium | To 100 | 4.79 |
| | polymethacrylate | chloride | | |
| | (MW = 4000-6000 | | | |
| | g/mol) | | | |
| 6 | 1% sodium | 0.5% calcium | To 100 | 4.52 |
| | polyacrylate | chloride | | |
| | (MW = 8000 g/mol) | | | |

The composition of model ionic sweat (pH 6.9) according to the formulation detailed in Table 2 are prepared:

TABLE 2

| Ingredient | wt % of total |
|---|---|
| Potassium Chloride | 0.0373 |
| Sodium Bicarbonate | 0.2025 |
| Sodium Chloride | 0.2098 |
| Ammonium Chloride | 0.0107 |
| Calcium Chloride | 0.0222 |
| Lactic Acid | 0.0901 |
| Urea | 0.0018 |
| Water | 99.4256 |

### Experiment Methods

An experiment was arranged using the device as disclosed in the International Application WO2018099931 A1 (Unilever).

The device has a 20×20 μm channel. Before use, the channel was filled with model ionic sweat. The surface tension of the model ionic sweat is strong and prevents its flowing in the capillary. 20 μl of concerned composition in Table-2, was placed at one end of the channel. When the composition contacts with the model ionic sweat, the composition of the present invention is believed to turn into something like an aggregation/agglomeration. This phenomenon simulates the manner in which an active agent forms aggregation/agglomeration thereby provides benefit of preventing sweat from rapidly being released, absorbing or managing excess sweat in the underarm region.

The whole process was monitored under optical microscope (Leica™ DM 2500P).

The data on the aggregation/agglomeration formation of the compositions at the end of the channel after 60 minutes is summarized in Table 3 below:

TABLE 3

| Samples | A | B | C | 1 | 2 | 3 | 4 | 5 |
|---|---|---|---|---|---|---|---|---|
| Aggregation/ | N | N | N | Y | Y | Y | Y | Y |
| agglomeration | | | | | | | | |
| formed | | | | | | | | |

The results (shown in the Table 3) demonstrate that compositions as per the invention (Reference No. 1 to 5) are capable of forming an aggregation/precipitation under the test conditions disclosed earlier, which further indicates their ability to prevent sweat from rapidly being released, absorb or manage excess sweat when used in a cosmetic composition, while compositions outside the invention (Examples A to C) do not exhibit such efficacy.

It was further observed that with the increase of dosage of polymer within the scope of the invention in the composition, there were more aggregation/agglomeration formed, thereby indicating stronger ability to prevent sweat from rapidly being released, absorb or manage excess sweat when used in a cosmetic composition.

1. An aqueous cosmetic composition comprising:
(i) a polymer selected from polyacrylic acid, polyacrylate, poly methacrylic acid and polymethacrylate;
(ii) a water-soluble metal salt wherein the metal is selected from calcium, magnesium, and zinc;
wherein the polymer has a molecular weight that is not higher than 8000 g/mol; wherein the composition has a pH ranging from 3 to 5.5; and
further wherein the composition comprises at least 1% of the polymer by weight of the composition.

2. A composition as claimed in claim 1 wherein the molecular weight of the polymer is from 2000 to 8000 g/mol.

3. A composition as claimed in claim 1 wherein the polymer is a polyacrylate, selected from sodium polyacrylate, potassium polyacrylate, ammonium polyacrylate, and lithium polyacrylate.

4. A composition as claimed in claim 1 wherein the polymer is a polymethacrylate, selected from sodium polymethacrylate, potassium polymethacrylate, ammonium polymethacrylate, and lithium polymethacrylate.

5. A composition as claimed in claim 1 wherein the composition comprises 1 to 20% of the polymer by weight of the composition.

6. A composition as claimed in claim 1 wherein composition comprises from 0.5 to 10% of the metal salt by weight of the composition.

7. A composition as claimed in claim 1 wherein pH of the composition is from 3 to 5.

8. A composition as claimed in claim 1 wherein the composition comprises 5 to 98% of water by weight of the composition.

9. A composition as claimed in claim 1 wherein the composition comprises a fragrance.

10. A composition as claimed in claim 1 wherein the composition comprises an antimicrobial deodorant active.

11. A composition as claimed in claim 1 wherein the composition is free from antiperspirant active which comprises aluminium or zirconium.

12. A composition as claimed in claim 1 wherein said composition is in the form of a spray, firm solid, soft solid, cream, or is an emulsion packaged in a roll-on applicator.

13. A composition as claimed in claim 1 when said composition is a spray it comprises a propellant and is in the form of an aerosol.

14. A method of preventing sweat from rapidly being released, absorbing or managing excess sweat comprising a step of topical application of the composition as claimed in claim 1 on to the desired skin surface.

15. (canceled)

16. A composition as claimed in claim 1 wherein the composition comprises from 2 to 15% of the polymer by weight of the composition.

17. A composition as claimed in claim 1 wherein the composition comprises from 4 to 10% of the polymer by weight of the composition.

18. A composition as claimed in claim 1 wherein composition comprises from 1 to 8% of the metal salt by weight of the composition.

19. A composition as claimed in claim 1 wherein composition comprises from 1.5 to 5% of the metal salt by weight of the composition.

20. A composition as claimed in claim 1 wherein pH of the composition is from 3.5 to 4.5.

21. A composition as claimed in claim 1 wherein the composition comprises from 15 to 95% of water by weight of the composition.
